(19) United States
(12) Patent Application Publication
(10) Pub. No.: US 2024/0033382 A1
(43) Pub. Date: Feb. 1, 2024

(54) METHOD FOR REDUCING BIOBURDEN OF CYCLIC LIPOPEPTIDE COMPOUND

(71) Applicant: KANEKA CORPORATION, Osaka (JP)

(72) Inventor: Noriyuki Kizaki, Hyogo (JP)

(73) Assignee: KANEKA CORPORATION, Osaka (JP)

(21) Appl. No.: 18/482,488

(22) Filed: Oct. 6, 2023

Related U.S. Application Data
(63) Continuation of application No. PCT/JP2022/012598, filed on Mar. 18, 2022.

(30) Foreign Application Priority Data
Apr. 8, 2021 (JP) 2021-066100

(51) Int. Cl. A61L 2/08; C07K 7/64

(52) U.S. Cl. CPC A61L 2/087; C07K 7/64

(57) ABSTRACT

A means of reducing bioburden in cyclic lipopeptide compound products by a simple operation without impairing their quality or function is provided.

A method for reducing bioburden of a cyclic lipopeptide compound is characterized by irradiating a cyclic lipopeptide compound with a radiation beam. A method for producing a cyclic lipopeptide compound with reduced bioburden includes irradiating a cyclic lipopeptide compound with a radiation beam.

### TECHNICAL FIELD

One or more embodiments of the present invention relate to a method for reducing bioburden of a cyclic lipopeptide compound.

### BACKGROUND

Cyclic lipopeptide compounds represented by surfactins and iturins are amphipathic substances originating from microorganisms and exhibit an excellent antibacterial or antifungal action against a wide range of bacteria or fungi, as well as a so-called surfactant action (Non Patent Literature 1). Hence, cyclic lipopeptide compounds are expected to be used in various fields such as medicine, food production, agriculture, and environmental hygiene as antibacterial agents, antifungal agents, therapeutic agents for infectious diseases, plant disease control agents, and the like.

Especially in the fields of manufacturing products for these applications, bioburden (the number of viable microorganisms contained in a unit amount of an object) significantly impacts product quality and safety. Bioburden control is therefore essential to ensure that the manufacturing environment and products are hygienically controlled.

Cyclic lipopeptide compounds such as surfactins and iturins are produced mainly by spore-forming bacteria of the genus Bacillus in culture (Patent Literature 1). In the process of purifying these compounds from the culture solutions, viable microorganisms cannot be removed entirely, resulting in contamination of products with viable microorganisms. Methods such as heat sterilization and filtration sterilization can be taken to reduce the number of viable microorganisms. However, since heating at a high temperature for a long time that can inactivate spores is a stringent condition, there are concerns about the burden of energy costs and the impact on quality. In addition, the operation for filtration sterilization is complicated and hardly applicable to iturins having poor solubility. Therefore, a method for reducing bioburden of a cyclic lipopeptide compound with a more simplified operation without affecting the quality has been desired.

### PATENT LITERATURE

Patent Literature 1: JP Patent No. 3635638

### Non Patent Literature

Non Patent Literature 1: Soil Microorganisms, Vol. 66, No. 1, p. 27 (2012)

### SUMMARY

One or more embodiments of the present invention provide means of reducing bioburden in cyclic lipopeptide compound products useful in various fields by a simple operation without impairing their quality or function.

As a result of intensive studies to solve the above, the present inventors found that bioburden can be significantly reduced by irradiating a surfactin or an iturin with a radiation beam, and besides, the irradiation causes no effects on their purity or functional activity. This has led to the completion of one or more embodiments of the present invention.

Specifically, one or more embodiments of the present invention encompass the following.

(1) A method for reducing bioburden of a cyclic lipopeptide compound, characterized by irradiating a cyclic lipopeptide compound with a radiation beam.

(2) A method for producing a cyclic lipopeptide compound with reduced bioburden, comprising irradiating a cyclic lipopeptide compound with a radiation beam.

(3) The method according to (1) or (2), wherein the cyclic lipopeptide compound is a surfactin, an iturin, a fengycin, or a salt thereof.

(4) The method according to any one of (1) to (3), wherein a purity of the cyclic lipopeptide compound is 50% by weight or more.

(5) The method according to any one of (1) to (4), wherein the radiation beam is a gamma-ray beam or an electron beam.

(6) The method according to any one of (1) to (5), wherein an absorbed radiation dose of the radiation beam is 100 kGy or less.

The present description includes the contents as disclosed in the description and/or drawings of Japanese Patent Application No. 2021-066100 filed on Apr. 8, 2021, which is a priority document of the present application.

According to the method of one or more embodiments of the present invention, it is possible to produce a cyclic lipopeptide compound, whereby bioburden has been reduced to a practically acceptable level. Further, the method of one or more embodiments of the present invention does not require a complicated operating procedure because the irradiation step is only performed at the end of the conventional method for producing a cyclic lipopeptide. Furthermore, the cyclic lipopeptide obtained by the method of one or more embodiments of the present invention has a significantly reduced bioburden, sufficiently retains functionality such as surfactant activity and antibacterial activity, does not change the appearance, emit odor, and is of extremely high quality.

### BRIEF DESCRIPTION OF THE DRAWINGS

FIG. 1 is a view showing the surfactant action (surface tension reducing action) of surfactin sodium salt irradiated with a gamma-ray beam to each absorbed radiation dose ((a): Before irradiation; (b): Irradiation at 8 kGy; (c): Irradiation at 15 kGy; (d): Irradiation at 30 kGy; (e): Irradiation at 60 kGy).

### DETAILED DESCRIPTION OF THE EMBODIMENTS

### Cyclic Lipopeptide Compound

The cyclic lipopeptide compound according to one or more embodiments of the present invention is a cyclic peptide compound acylated with a fatty acid and is produced by microorganisms such as bacteria belonging to the genus Bacillus. Examples of cyclic lipopeptide compounds include surfactins, iturins, and fengycins, which may be in the form of a mixture of one or more thereof. These cyclic lipopeptides may also be in the salt form.

A surfactin is a cyclic lipopeptide composed of seven amino acids linked to a (β-hydroxy fatty acid. Specific examples thereof include a compound represented by the following formula (I) and a mixture containing two or more compounds represented by the formula (I). For example, it may be a mixture of a plurality of compounds (I) that differ in the number of carbon atoms in group R.

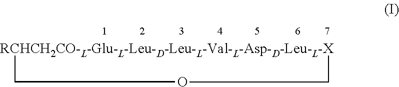

In Formula (I), X represents an amino acid residue selected from the group consisting of leucine, isoleucine, valine, glycine, serine, alanine, threonine, asparagine, glutamine, aspartic acid, glutamic acid, lysine, arginine, cysteine, methionine, phenylalanine, tyrosine, tryptophan, histidine, proline, 4-hydroxyproline, and homoserine. X may be leucine, isoleucine, or valine.

In Formula (I), R represents a normal alkyl group having 8 to 14 carbon atoms, an isoalkyl group having 8 to 14 carbon atoms, or an anteisoalkyl group having 8 to 14 carbon atoms. A normal alkyl group is a linear alkyl group. An isoalkyl group generally has a structure that includes (CH₃)₂CH—(CH₂)ₙ—. An anteisoalkyl group generally has a structure that includes CH₃—CH₂—CH(CH₃)—(CH₂)ₙ—.

The surfactin may be a surfactin analog in which one or more amino acids in Formula (I) are substituted with other amino acids. Examples thereof include compounds in which the amino acid at the second position, namely L-leucine, the amino acid at the fourth position, namely L-valine, and the amino acid at the sixth position, namely D-leucine, are independently substituted with an amino acid selected from the group consisting of leucine, isoleucine, valine, glycine, serine, alanine, threonine, asparagine, glutamine, aspartic acid, glutamic acid, lysine, arginine, cysteine, methionine, phenylalanine, tyrosine, tryptophan, histidine, proline, 4-hydroxyproline, and homoserine.

The surfactin may be an inorganic or organic salt of the compound represented by Formula (I). In a case in which the surfactin is an inorganic salt, a metal that is a counter ion is not particularly limited as long as the counter ion can form a salt with a surfactin. Examples of the counter ion include: alkaline metals such as sodium, potassium, and lithium; and alkaline earth metals such as calcium and magnesium. In a case in which the surfactin is an organic salt, examples thereof include a trimethylamine salt, a triethylamine salt, a tributylamine salt, a monoethanolamine salt, a diethanolamine salt, a triethanolamine salt, a lysine salt, an arginine salt, and a choline salt.

Among the above-described salts, a sodium salt, a potassium salt, a monoethanolamine salt, a diethanolamine salt, a triethanolamine salt, a lysine salt, and an arginine salt are preferable, and a sodium salt is particularly preferable.

An iturin is a cyclic lipopeptide composed of seven amino acids linked to a (β-amino fatty acid. Examples thereof include iturin A, iturin A1, iturin C, bacillomycin A, bacillomycin B, bacillomycin C, bacillomycin D, bacillomycin F, bacillomycin L, bacillomycin LC, and mycosubtilin. The number of carbon atoms of the (β-amino fatty acid that constitutes an iturin is, for example, 12 to 18. Iturin A, iturin A1, iturin C, bacillomycin A, bacillomycin B, bacillomycin C, bacillomycin D, bacillomycin F, bacillomycin L, bacillomycin LC, mycosubtilin, and the like may be in the form of a mixture of a plurality of compounds in which the number of carbon atoms of the (β-amino fatty acids that constitute them differ from each other.

A fengycin is a cyclic lipopeptide composed of 10 amino acids linked to a (β-hydroxy fatty acid. Examples thereof include fengycin A, fengycin B, plipastatin A, and plipastatin B. The number of carbon atoms of the (β-hydroxy fatty acid that constitutes a fengycin is, for example, 14 to 21. Fengycin A, fengycin B, plipastatin A, plipastatin B, and the like may be in the form of a mixture of a plurality of compounds in which the number of carbon atoms of the (β-hydroxy fatty acid that constitute them differs from each other.

The above-described cyclic lipopeptides can be produced by culturing a microorganism capable of producing them, for example, a bacterium belonging to the genus Bacillus to produce and accumulate a cyclic lipopeptide in a culture solution, after which the cyclic lipopeptide is recovered from the culture solution and purified. Microorganisms to be used are not particularly limited as long as they have the ability to produce the cyclic lipopeptides, and those known to a person skilled in the art can be used. Examples of bacteria of the genus Bacillus include Bacillus subtilis, Bacillus amyloliquefaciens, Bacillus cereus, Bacillus thuringiensis, Bacillus coagulans, Bacillus pumilus, and Bacillus licheniformis.

Culture may be performed according to known methods and conditions. For instance, in producing a surfactin in the most simple manner, for example, Bacillus subtilis SD901 (FERM BP-7666) may be cultured in a nutrient medium such as L-medium containing 10 ppm tetracycline at 25° C. to 42° C., 28° C. to 40° C., or 30° C. to 37° C. for about 5 to 24 hours, and the resulting culture solution may be inoculated into a medium containing an appropriate nitrogen source at 0.1% to 10% (w/w), 0.5% to 7% (w/w), or 1% to 5% (w/w). This may be cultured at 25° C. to 42° C., 28° C. to 40° C., or 30° C. to 37° C. for about 20 to 90 hours.

Purification can be performed by, for example, acidifying the culture solution by adding sulfuric acid, hydrochloric acid, nitric acid, or the like and filtering the precipitated cyclic lipopeptide and dissolving the precipitates in an organic solvent such as methanol, followed by ultrafiltration, activated carbon treatment, crystallization, or the like.

### Irradiation

In one or more embodiments of the present invention, a cyclic lipopeptide is irradiated with a radiation beam, for example, after culturing a bacterium of the genus Bacillus as described above and collecting a cyclic lipopeptide produced and accumulated in a culture solution from the culture solution. Therefore, it can be said that the method for reducing bioburden of a cyclic lipopeptide compound of one or more embodiments of the present invention is also a method for producing a cyclic lipopeptide compound with reduced bioburden.

The purity of a cyclic lipopeptide compound to be irradiated with a radiation beam is not particularly limited as long as the effects of one or more embodiments of the present invention can be obtained. It may be, for example, 50% by weight or more, 70% by weight or more, 80% by weight or more, 90% by weight or more, or 95% by weight or more. Irradiation may be performed before or after filling a cyclic lipopeptide into a container such as a bag or a bottle.

Radiation beams as used in one or more embodiments of the present invention refer to high-energy particle beams and electromagnetic waves. Examples thereof include an alpha-ray beam, a beta-ray beam, a gamma-ray beam, a neutron beam, an electron beam, and an X-ray beam. Of these, a gamma-ray beam or an electron beam is preferable, and a gamma-ray beam is more preferable. The absorbed radiation dose of a radiation beam necessary to obtain the effects of one or more embodiments of the present invention can be determined based on the number of viable microorganisms (bioburden) contained per unit weight of a target cyclic lipopeptide compound, the D value (decimal reduction value), which is the absorbed radiation dose required to reduce the number of the viable microorganisms to 1/10, and the desired bioburden level after irradiation. In the case of a gamma-ray beam or an electron beam, for example, 5 kGy or more can be exemplified. However, as the absorbed radiation dose increases, the target cyclic lipopeptide compound can be denatured and altered. In view of this, for example, in the case of a gamma-ray beam or an electron beam, the absorbed radiation dose may be 100 kGy or less, 60 kGy or less, 30 kGy or less, or 15 kGy or less. The temperature during irradiation is not particularly limited as long as the effects of one or more embodiments of the present invention can be obtained. It is, for example, −20° C. to 80° C. From the viewpoint of carrying out an irradiation operation in a simple manner, around room temperature is preferable.

### Bioburden

As used herein, “bioburden” refers to the “number of viable microorganisms contained in a unit amount of a cyclic lipopeptide compound product.” As used herein, viable microorganisms are bacteria and fungi. In addition, “reducing bioburden” refers to “reducing the number of viable microorganisms at a detectable level.” The number of microorganisms can be measured, for example, according to the method described in the 17th revised Japanese Pharmacopoeia “4.05 Microbiological Examination of Non-sterile Products, I. Microbiological Examination of Non-sterile Products: Microbial Enumeration Tests” (the method for measuring the number of colonies observed after culturing at 35° C. for 3 days under aerobic conditions using an SCD agar medium).

### EXAMPLES

One or more embodiments of the present invention will be specifically described in the following examples. However, one or more embodiments of the present invention are not limited to these examples.

### (Example 1) Irradiation of Surfactin with Gamma-Ray Beam

### (1) Preparation of Surfactin Sodium Salt

A surfactin sodium salt was prepared from a culture medium of Bacillus subtilis SD901 (FERM BP-7666) according to the method described in Example 6 of JP Patent No. 5868325. Three lots of the surfactin sodium salt were prepared. Each lot (A, B, C) of the surfactin sodium salt was subjected to HPLC analysis under the following conditions. Quantitative determination was performed based on a calibration curve prepared using a surfactin standard sample (manufactured by Sigma-Aldrich), thereby calculating quantity of pure surfactin sodium salt in each of the lot (A, B, C).

### HPLC Conditions

Sample volume: 20 μL

Column: ODS-2, 4.6 mm×250 mm, manufactured by GL Science

Column temperature: 40° C.

Eluent: 80% (v/v) acetonitrile, 3.8 mM trifluoroacetic acid

Flow rate: 1.5 mL/min

Detector: UV detector

Wavelength: 205 nm

As a result, their purities were 87.6% by weight for lot A, 93.1% by weight for lot B, and 96.7% by weight for lot C.

### (2) Determination of Viable Cell Count in Surfactin Sodium Salt

10 mL of aqueous solution was prepared by dissolving 1 g of the surfactin sodium salt (lot A) prepared in (1) in sterile water. This aqueous solution was serially diluted, and 0.1 mL each was smeared on three plates of SCD agar medium, and the number of colonies that appeared after culturing at 35° C. for 3 days was counted. A dilution rate suitable for colony counting was selected. The number obtained by multiplying the average number of colonies appearing on the three plates by the dilution rate and then further by 100 was defined as the number of viable cells contained in 1 g of a surfactin sodium salt sample (unit: cfu/g). As a result, the viable cell count in the surfactin sodium salt (lot A) was 1.6×10⁶ cfu/g.

(3) Irradiation of Surfactin Sodium Salt with Gamma-ray Beam

The lot A of the surfactin sodium salt prepared in (1) was filled in two sterilized polyethylene bags and irradiated with a gamma-ray beam using cobalt 60 as a radiation source, thereby yielding an absorbed radiation dose of 8 kGy or 15 kGy. The number of viable cells in the surfactin sodium salt after gamma-ray beam irradiation was measured in the same manner as in (2). As a result, after irradiation at 8 kGy, it decreased to 6.0×10² cfu/g, and after irradiation at 15 kGy, it declined to as low as less than 1×10² cfu/g, which is the detection limit. As described in (2), the viable cell count before gamma-ray beam irradiation was 1.6×10⁶ cfu/g, and it was confirmed that the viable cell count decreased exponentially as the absorbed radiation dose increased.

### (4) Influence of Gamma-ray Beam Irradiation on Purity

Each lot (A, B, C) of the surfactin sodium salt prepared in (1) was irradiated with a gamma-ray beam in the same manner as in (3), thereby yielding absorbed radiation doses of 8 kGy, 15 kGy, 30 kGy, and 60 kGy. The purity of each surfactin sodium salt after gamma-ray beam irradiation was measured in the same manner as in (1) and the results are shown in Table 1 below.

TABLE 1
Purity of surfactin sodium salt before
and after gamma-ray beam irradiation

| | Sample | | |
|---|---|---|---|
| Dose (kGy) | Lot A | Lot B | Lot C |
| Before irradiation | 87.6 | 93.1 | 96.7 |
| 8 | 87.8 | 93.0 | 96.7 |
| 15 | 87.7 | 92.9 | 96.5 |
| 30 | 87.6 | 93.1 | 96.7 |
| 60 | 87.5 | 93.0 | 96.5 |

(Unit: % wt)

As shown in Table 1, no decrease in purity was observed after every lot of the surfactin sodium salt was irradiated with a gamma-ray beam. In addition, no change in appearance (color tone) and odor was observed due to gamma-ray beam irradiation.

### (5) Critical Micelle Concentration Measurement of Surfactin Sodium Salt

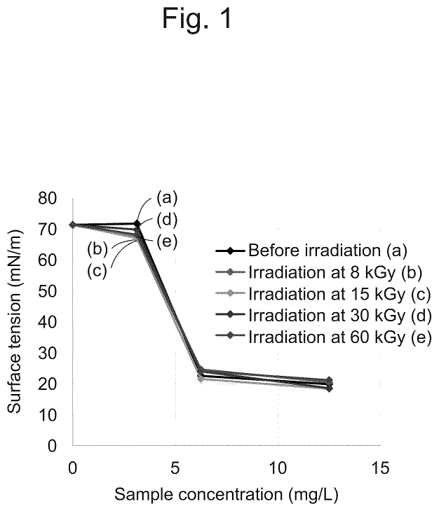

To examine the critical micelle concentration before and after irradiation, the lot C of the surfactin sodium salt prepared in (1) was irradiated with a gamma-ray beam in the same manner as in (3), thereby yielding absorbed radiation doses of 8 kGy, 15 kGy, 30 kGy, and 60 kGy, and then the aqueous solution of each sample prepared at a concentration of 100 mg/L was diluted twice in sequence, thereby preparing aqueous solutions having concentrations of 12.5 mg/L, 6.25 mg/L, and 3.125 mg/L. FIG. 1 shows the results of measuring the surface tension for these aqueous solutions and water as a control by the Wilhelmy method. For each sample, the surface tension decreased significantly between concentrations of 3.125 mg/L and 6.25 mg/L. Therefore, it was considered that the critical micelle concentration exists in this range. Accordingly, no changes due to gamma-ray beam irradiation were confirmed. From the above, it was found that gamma-ray beam irradiation does not influence the function of the surfactin sodium salt as a surfactant.

### (Example 2) Irradiation of Iturin with Gammar-ray Beam

### (1) Preparation of Iturin

5 L of medium having the following composition was prepared in a 10 L volume culture tank, inoculated with Bacillus subtilis capable of producing an iturin, and cultured at 35° C. for 72 hours.

### Medium Composition

Soybean flour: 8% by weight

K₂HPO₄: 0.5% by weight

MgSO₄·7H₂O: 0.05% by weight

FeSO₄·7H₂O: 0.0025% by weight

MnSO₄·5H₂O: 0.0022% by weight

CaCl₂: 0.0184% by weight

Maltose: 6.7% by weight

Balance: Water

The resulting culture solution was extracted with 1-butanol, and 1-butanol in the separated organic phase was distilled off and replaced with water, thereby obtaining a suspension containing an iturin. Ethyl acetate was added thereto, and the mixture was stirred, centrifuged to collect a precipitate, and further dried under reduced pressure, thereby obtaining a crudely purified iturin. The purity of the crudely purified iturin was 71.9%. The iturin was quantitatively determined by HPLC under the following conditions using an iturin manufactured by Sigma-Aldrich as a reference standard.

### HPLC Conditions

Sample volume: 20 μL

Column: Shodex Silica C18P4E, 4.6 mm×250 mm, manufactured by Showa Denko K.K.

Column temperature: 40° C.

Eluent: acetonitrile:10 mM ammonium acetate aqueous solution=35:75 (v/v)

Flow rate: 1.5 mL/min

Detector: UV detector

Wavelength: 205 nm

### (2) Influence of Gamma-ray Beam Irradiation on Purity

The crudely purified iturin prepared in (1) was filled in two sterilized polyethylene bags and irradiated with a gamma-ray beam using cobalt 60 as a radiation source, thereby yielding an absorbed radiation dose of 30 kGy or 60 kGy. The crudely purified iturin after gamma-ray beam irradiation was measured in the same manner as in (1) and the results are shown in Table 2 below.

TABLE 2

| Dose (kGy) | Purity (% wt) |
|---|---|
| Before irradiation | 71.9 |
| 30 | 71.6 |
| 60 | 71.7 |

As shown in Table 2, no decrease in the purity of the iturin was observed even after gamma-ray beam irradiation at each of the doses. In addition, no change in appearance (color tone) and odor was observed due to gamma-ray beam irradiation.

(3) Antifungal Activity of Iturin Irradiated with Gamma-ray Beam

Fifty milligrams (50 mg) of each of the crudely purified iturin prepared in (1) and the crudely purified iturin prepared in (2) after gamma-ray beam irradiation were dissolved in 5 mL of dimethylsulfoxide. These solutions were added to potato dextrose agar medium such that the final concentration of each crudely purified iturin was 5 mg/L and 30 mg/L, thereby preparing plate media. In the center of each of these plate media, a mycotic lawn disc obtained by punching test fungi (Alternaria mali, Fusarium oxysporum, Rhizoctonia solani) listed in Table 3, which were preliminarily plate-cultured on a potato dextrose agar medium, to a diameter of 5 mm was placed and cultured at 25° C. for 48 hours. The radius of mycotic lawn of each test fungi grown on each plate medium was measured, and the mycelium elongation inhibition rate was calculated by the following formula.

Mycelium elongation inhibition rate (%)=[(Radius of mycotic lawn in medium without added iturin−Radius of mycotic lawn in medium with added iturin)/Radius of mycotic lawn in medium without added iturin]×100

The results are shown in Table 3 below.

TABLE 3
Mycelium elongation inhibition rate of iturin irradiated with gamma-ray beam

| | | Dose | | | | | |
|---|---|---|---|---|---|---|---|
| | | Before irradiation | | 30 kGy | | 60 kGy | |
| | | Concentration added to medium | | | | | |
| | | 5 mg/L | 30 mg/L | 5 mg/L | 30 mg/L | 5 mg/L | 30 mg/L |
| Test | Alternaria mali | 16 | 42 | 15 | 43 | 18 | 40 |
| fungi | Fusarium oxysporum | 23 | 55 | 23 | 53 | 21 | 56 |
| | Rhizoctonia solani | 48 | 77 | 49 | 79 | 46 | 76 |

(Unit: %)

As shown in Table 3, no difference was observed in the mycelium elongation inhibition rate of the iturin between the presence and absence of gamma-ray beam irradiation, and the iturin maintained the same antifungal activity even after gamma-ray beam irradiation.

### (4) Reduction of Number of General Bacteria in Iturin by Gamma-ray Beam Irradiation

Fifty milligrams (50 mg) of each of the crudely purified iturin prepared in (1) and the crudely purified iturin prepared in (2) after gamma-ray beam irradiation were dissolved in 5 mL of dimethylsulfoxide. Each of these solutions was serially diluted with sterile water, and 0.1 mL each was smeared on three plates of SCD agar medium, and the number of colonies that appeared after culturing at 35° C. for 3 days was counted. A dilution rate suitable for colony counting was selected. The number obtained by multiplying the average number of colonies appearing on the three plates by the dilution rate and then further by 1000 was defined as the number of general bacteria contained in 1 g of an iturin sample (unit: cfu/g). As a result, the number of general bacteria in the crudely purified iturin prepared in (1) was 7.1×10⁴ cfu/g. On the other hand, regarding the crudely purified iturin irradiated with a gamma-ray beam in (2), no colonies appeared even when 0.1 mL of the above-described dimethylsulfoxide solution was smeared onto the plate without dilution, and the number of general bacteria was as less than 10³ cfu/g, which is the detection limit.

One or more embodiments of the present invention can be used in the field of manufacturing pharmaceuticals, foods, agricultural chemicals, and the like using cyclic lipopeptides.

All publications, patents, and patent applications cited in the present description are incorporated herein by reference in their entirety.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

1. A method for reducing bioburden of a cyclic lipopeptide compound, characterized by irradiating a cyclic lipopeptide compound with a radiation beam.

2. A method for producing a cyclic lipopeptide compound with reduced bioburden, comprising irradiating a cyclic lipopeptide compound with a radiation beam.

3. The method according to claim 1, wherein the cyclic lipopeptide compound is a surfactin, an iturin, a fengycin, or a salt thereof.

4. The method according to claim 1, wherein a purity of the cyclic lipopeptide compound is 50% by weight or more.

5. The method according to claim 1, wherein the radiation beam is a gamma-ray beam or an electron beam.

6. The method according to claim 1, wherein an absorbed radiation dose of the radiation beam is 100 kGy or less.

7. The method according to claim 2, wherein the cyclic lipopeptide compound is a surfactin, an iturin, a fengycin, or a salt thereof.

8. The method according to claim 2, wherein a purity of the cyclic lipopeptide compound is 50% by weight or more.

9. The method according to claim 2, wherein the radiation beam is a gamma-ray beam or an electron beam.

10. The method according to claim 2, wherein an absorbed radiation dose of the radiation beam is 100 kGy or less.
